(19) United States
(12) Patent Application Publication
(10) Pub. No.: US 2013/0165823 A1
(43) Pub. Date: Jun. 27, 2013

(54) ULTRASONIC IRRADIATION DEVICE

(71) Applicant: OLYMPUS CORPORATION, Tokyo (JP)

(72) Inventors: Yoshiharu ISHIBASHI, Hachioji-shi (JP); Miyuki MURAKAMI, Hachioji-shi (JP); Hiroshi TSURUTA, Hachioji-shi (JP)

(73) Assignee: OLYMPUS CORPORATION, Tokyo (JP)

(21) Appl. No.: 13/771,541

(22) Filed: Feb. 20, 2013

Related U.S. Application Data
(63) Continuation of application No. PCT/JP2011/074835, filed on Oct. 27, 2011.

(30) Foreign Application Priority Data
Oct. 27, 2010 (JP) 2010-240789

(51) Int. Cl. A61N 7/00

(52) U.S. Cl. CPC A61N 7/00
USPC 601/2

(57) ABSTRACT

An ultrasonic irradiation device includes a sound source, a holding member, a first support member and a second support member. The sound source is configured to be placed in a space at least a part of which is covered with a wall surface and emit an ultrasonic wave toward a target area. The holding member is configured to hold the sound source. The first support member is configured to push a first area of the wall surface so that a distance between the sound source and the target area is kept at a predetermined value, the first supporting member being disposed on the holding member. The second support member is configured to push a second area of the wall surface including an area other than the first area so that the sound source is fixed to the wall surface together with the first support member.

### CROSS-REFERENCE TO RELATED APPLICATIONS

This application is a Continuation application of PCT Application No. PCT/JP2011/074835, filed Oct. 27, 2011 and based upon and claiming the benefit of priority from prior Japanese Patent Application No. 2010-240789, filed Oct. 27, 2010, the entire contents of all of which are incorporated herein by reference.

### BACKGROUND OF THE INVENTION

1. Field of the Invention

The present invention relates to an ultrasonic irradiation device.

2. Description of the Related Art

There is known a treatment by which a living organism is irradiated with a focused ultrasonic wave to cauterize an affected area of, for example, cancer cells. For carrying out such a treatment in the living body, an ultrasonic irradiation device is known in which the focused ultrasonic source is disposed at an insertion portion of an endoscope, the insertion portion being inserted into the living body. A technique concerning such an ultrasonic irradiation device is disclosed in, for example, Japanese Patent No. 3850094. According to this technique, in the ultrasonic irradiation device in which the focused ultrasonic source is disposed in the endoscope, balloons are attached to positions in the vicinity of the ultrasonic source. The sizes of the balloons determine a distance between the focused ultrasonic source and a wall surface of an ultrasonic irradiation target. That is, they determine a distance between a focal point of the focused ultrasonic wave and a target position such as an affected area which is to be irradiated with the ultrasonic wave.

### BRIEF SUMMARY OF THE INVENTION

According to an aspect of the present invention, an ultrasonic irradiation device includes a sound source configured to be placed in a space at least a part of which is covered with a wall surface and emit an ultrasonic wave toward a target area; a holding member configured to hold the sound source; a first support member configured to push a first area of the wall surface so that a distance between the sound source and the target area is kept at a predetermined value, the first supporting member being disposed on the holding member; and a second support member configured to push a second area of the wall surface including an area other than the first area so that the sound source is fixed to the wall surface together with the first support member.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing an example of a configuration of an ultrasonic irradiation device according to a first embodiment of the present invention;

FIG. 2 is a schematic view for explaining movement of an inserting portion of the ultrasonic irradiation device according to the first embodiment of the present invention;

FIG. 3 is a view showing an example of a configuration of a probe part of an ultrasonic irradiation device according to a second embodiment of the present invention;

FIG. 4 is a schematic view for explaining movement of an inserting portion of the ultrasonic irradiation device according to the second embodiment of the present invention;

FIG. 5 is a view showing an example of a configuration of a probe part of an ultrasonic irradiation device according to a third embodiment of the present invention;

FIG. 6 is a schematic view for explaining movement of an inserting portion of the ultrasonic irradiation device according to the third embodiment of the present invention;

FIG. 7 is a view showing an example of a configuration of a probe part of an ultrasonic irradiation device according to a fourth embodiment of the present invention;

FIG. 8 is a schematic view for explaining movement of an inserting portion of the ultrasonic irradiation device according to the fourth embodiment of the present invention;

FIG. 9 is a view showing another example of a configuration of the probe part of the ultrasonic irradiation device according to the fourth embodiment of the present invention;

FIG. 10 is a view showing an example of a configuration of a probe part of an ultrasonic irradiation device according to a fifth embodiment of the present invention;

FIG. 11 is a schematic view for explaining movement of an inserting portion of the ultrasonic irradiation device according to the fifth embodiment of the present invention; and

FIG. 12 is a block diagram showing an example of a configuration of an ultrasonic irradiation device according to a sixth embodiment of the present invention.

### DETAILED DESCRIPTION OF THE INVENTION

### First Embodiment

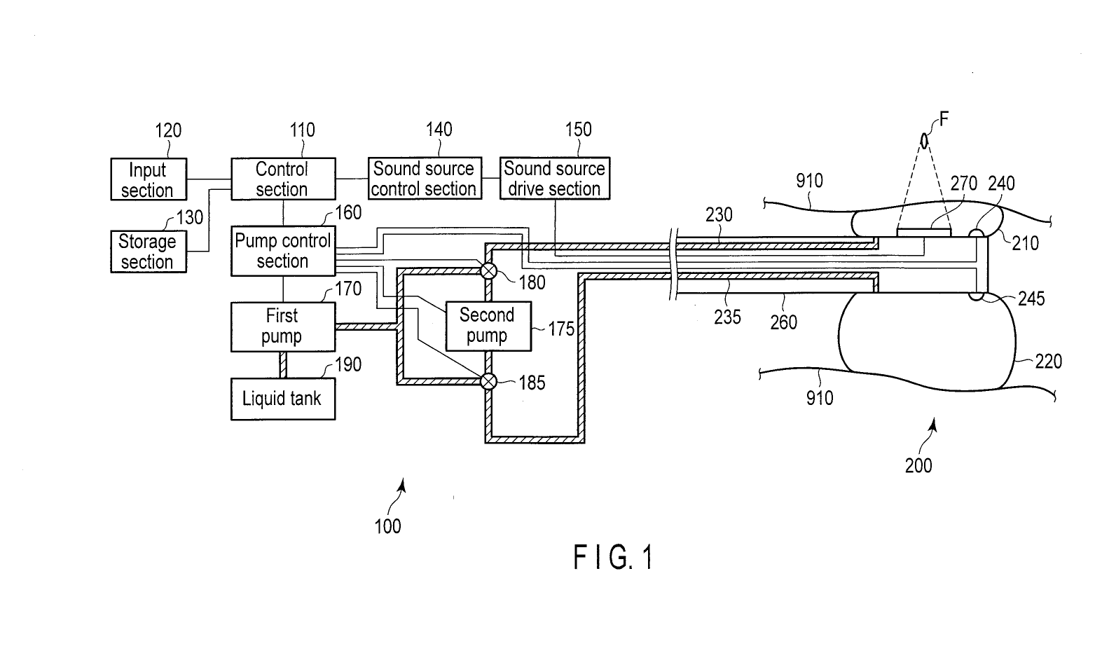

A first embodiment of the present invention will be described with reference to the drawings. An ultrasonic irradiation device according to the present embodiment is an endoscope type ultrasonic irradiation device which enables irradiation with a focused ultrasonic wave. For example, the present ultrasonic irradiation device is inserted into a living body, and irradiates a target position of a living organism (e.g., a tumor) with the focused ultrasonic wave to cauterize the organism. FIG. 1 is a block diagram showing a configuration of the ultrasonic irradiation device according to the first embodiment.

As shown in FIG. 1, the ultrasonic irradiation device according to the present embodiment includes a control part 100 which controls each section of the present ultrasonic irradiation device from the outside of a subject, and a probe part 200 which is configured to be inserted into the subject. The probe part 200 includes an elongate inserting portion 260 to be inserted into, for example, a stomach. A side of the inserting portion 260 which is to be inserted into the subject is called a distal side, and the control part 100 side of the inserting portion is called a proximal side. On a peripheral surface of the inserting portion 260 in the vicinity of the tip of the portion, a sound source 270 which emits the ultrasonic wave is disposed. The sound source 270 includes a piezoelectric element such as lead zirconate titanate (PZT). The surface of the sound source 270 which emits the ultrasonic wave has, for example, a concave surface shape, and the ultrasonic wave emitted from the sound source 270 becomes the focused ultrasonic wave converged on a focal point F.

In the vicinity of the sound source 270 of the inserting portion 260, a first balloon 210 and a second balloon 220 are arranged to fix the tip of the inserting portion 260 to, for example, the inside of the stomach. In the present embodiment, the first balloon 210 is expandably/contractibly disposed on a side of the ultrasonic emitting surface of the sound source 270. On the other hand, the second balloon 220 is expandably/contractibly disposed at a position where the second balloon faces the first balloon 210 via the inserting portion 260. The first balloon 210 and the second balloon 220 are made of an expandable/contractible material such as Latex rubber or another rubber.

The first balloon 210 is connected to a first tube 230. The first tube 230 is inserted through the inserting portion 260 and connected to the control part 100. A liquid is injected into and discharged from the first balloon 210 through the first tube 230. By the injection and discharge of the liquid, the first balloon 210 expands and contracts. Here, the liquid is, for example, physiological saline or deaerated water. Similarly, the second balloon 220 is connected to a second tube 235 which is inserted through the inserting portion 260 and connected to the control part 100. The liquid is injected into and discharged from the second balloon 220 through the second tube 235, and the second balloon 220 expands and contracts. Moreover, in the first balloon 210, a first pressure sensor 240 is disposed to measure a pressure of the liquid in the first balloon 210. Similarly, in the second balloon 220, a second pressure sensor 245 is disposed to measure a pressure of the liquid in the second balloon 220.

The control part 100 includes a control section 110, an input section 120, a storage section 130, a sound source control section 140, a sound source drive section 150, a pump control section 160, a first pump 170, a second pump 175, a first three-way valve 180, a second three-way valve 185, and a liquid tank 190. The control section 110 is connected to the input section 120, the storage section 130, the sound source control section 140, and the pump control section 160. The control section 110 controls the whole present ultrasonic irradiation device. The input section 120 is, for example, a keyboard into which an instruction from a user is input. The storage section 130 stores information on the control of the present ultrasonic irradiation device, and the like, and outputs the suitable information to the control section 110 in response to a request from the control section 110. The sound source control section 140 controls an output of the sound source 270, and the like. The sound source drive section 150 drives the sound source 270 under the control of the sound source control section 140.

The pump control section 160 controls operations of the first pump 170, the second pump 175, the first three-way valve 180 and the second three-way valve 185. Moreover, the pump control section 160 is connected to the first pressure sensor 240, and receives an output value of the first pressure sensor 240 to acquire the pressure of the liquid in the first balloon 210. Similarly, the pump control section 160 is connected to the second pressure sensor 245, and receives an output value of the second pressure sensor 245 to acquire the pressure of the liquid in the second balloon 220.

The liquid tank 190 which contains the liquid is connected to the first pump 170. The liquid tank 190 includes an unshown liquid temperature adjuster and an unshown deaerator. The first three-way valve 180 is connected to the first tube 230, the first pump 170, and the second pump 175. The second three-way valve 185 is connected to the second tube 235, the first pump 170, and the second pump 175.

The first pump 170 feeds the liquid from the liquid tank 190 toward the first three-way valve 180 and the second three-way valve 185, and conversely feeds the liquid toward the liquid tank 190 through the first three-way valve 180 and the second three-way valve 185. Therefore, when the first three-way valve 180 connects the first pump 170 to the first tube 230 and the second three-way valve 185 connects the first pump 170 to the second tube 235, the first pump 170 feeds the liquid from the liquid tank 190 toward the first tube 230 and the second tube 235, and conversely feeds the liquid toward the liquid tank 190 through the first tube 230 and the second tube 235. As a result, owing to the movement of the liquid, volumes of the first balloon 210 and the second balloon 220 can be changed.

The second pump 175 can feed the liquid in a direction from the first three-way valve 180 to the second three-way valve 185, and in the opposite direction. Therefore, when the first three-way valve 180 connects the second pump 175 to the first tube 230 and the second three-way valve 185 connects the second pump 175 to the second tube 235, the second pump 175 can feed the liquid in a direction from the first tube 230 to the second tube 235, and in the opposite direction. As a result, the liquid can be moved between the first balloon 210 and the second balloon 220. That is, a volume ratio between the first balloon 210 and the second balloon 220 can be changed. When the operations of the first pump 170, the second pump 175, the first three-way valve 180 and the second three-way valve 185 are controlled in this way, the volume of the first balloon 210 and the volume of the second balloon 220 can arbitrarily be regulated. Thus, in the present embodiment, the liquid to be filled into the first balloon 210 is the same as the liquid to be filled into the second balloon 220.

In consequence, for example, the inserting portion 260 functions as a holding member. For example, the sound source 270 functions as a sound source. For example, the first balloon 210 functions as a first support member. For example, the second balloon 220 functions as a second support member. For example, the first pump 170, the second pump 175, the first three-way valve 180 and the second three-way valve 185 function as a fluid adjusting section. For example, the pump control section 160 functions as a control section.

An operation of the ultrasonic irradiation device according to the present embodiment will be described. The user inserts the inserting portion 260 of the present ultrasonic irradiation device, for example, from a subject's mouth through the esophagus into the stomach. At this time, the liquid is not injected into the first balloon 210 and the second balloon 220, and the first balloon 210 and the second balloon 220 are contracted and received in the inserting portion 260. Therefore, the probe part 200 has, for example, a sufficiently slim configuration to pass through the esophagus. The user allows the ultrasonic emitting surface of the sound source 270 to face an area to be irradiated with the focused ultrasonic wave. In this state, the user inputs the instruction into the input section 120 of the present ultrasonic irradiation device to fix the probe part 200.

The input section 120 receives the user's instruction to fix the probe part 200, and outputs the instruction to the control section 110. The control section 110 outputs, to the pump control section 160, an instruction to start the control for fixing the probe part 200. The instruction is input into the pump control section 160 from the control section 110. The pump control section 160 controls the operations of the first pump 170, the second pump 175, the first three-way valve 180 and the second three-way valve 185, and injects the liquids into the first balloon 210 and the second balloon 220, to expand the first balloon 210 and the second balloon 220.

The first pump 170 under the control of the pump control section 160 feeds the suitably heated or cooled and suitably deaerated liquid contained in the liquid tank 190 toward the first tube 230 and the second tube 235. At this time, the second pump 175, the first three-way valve 180 and the second three-way valve 185 are regulated to adjust a size of the first balloon 210 and a size of the second balloon 220. As a result, while the position of the tip of the inserting portion 260 is maintained, the first balloon 210 and the second balloon 220 come in contact with a wall surface 910 such as a stomach wall. In these operations, the pump control section 160 acquires the pressure of the liquid in the first balloon 210 and the pressure of the liquid in the second balloon 220 on the basis of the output values of the first pressure sensor 240 and the second pressure sensor 245. After it is confirmed that the focal point F is aligned with and fixed to a target position, the pump control section 160 acquires the pressure of the liquid in the first balloon 210 and the pressure of the liquid in the second balloon 220, and then controls the operation of the second pump 175 so that these pressure values are maintained while the liquid is fed.

When the first balloon 210 and the second balloon 220 push the wall surface 910 with predetermined forces, the inserting portion 260 provided with the first balloon 210 and the second balloon 220 is fixed to the wall surface 910. In this case, since the force of the first balloon 210 to push the inserting portion 260 is equal to the force of the second balloon 220 to push the inserting portion 260, the position of the tip of the inserting portion 260 does not shift toward the first balloon 210 or the second balloon 220. In a configuration where a contact area between the first balloon 210 and the inserting portion 260 is always equal to a contact area between the second balloon 220 and the inserting portion 260, the pressure of the liquid in the first balloon 210 is always equal to the pressure of the liquid in the second balloon 220.

After confirming that the position to be irradiated with the focused ultrasonic wave is superimposed on the focal point F, the user inputs an instruction for ultrasonic irradiation into the input section 120. Additionally, the user can confirm a behavior that the inserting portion 260 is fixed to the wall surface 910, or the like by use of an ultrasonic diagnosis device which irradiates the outside of the living body with the ultrasonic wave to observe the inside of the living body, or the like.

The input section 120 into which the instruction for the ultrasonic irradiation has been input outputs this instruction to the control section 110. The control section 110 outputs, to the sound source control section 140, an instruction to allow the sound source to start the emission of the ultrasonic wave. The sound source control section 140 receives the instruction of the control section 110, determines a time, intensity or the like of the ultrasonic irradiation on the basis of this instruction, and controls the sound source drive section 150. The sound source drive section 150 drives the sound source 270 under the control of the sound source control section 140. The sound source 270 is driven by the sound source drive section 150 to emit the focused ultrasonic wave.

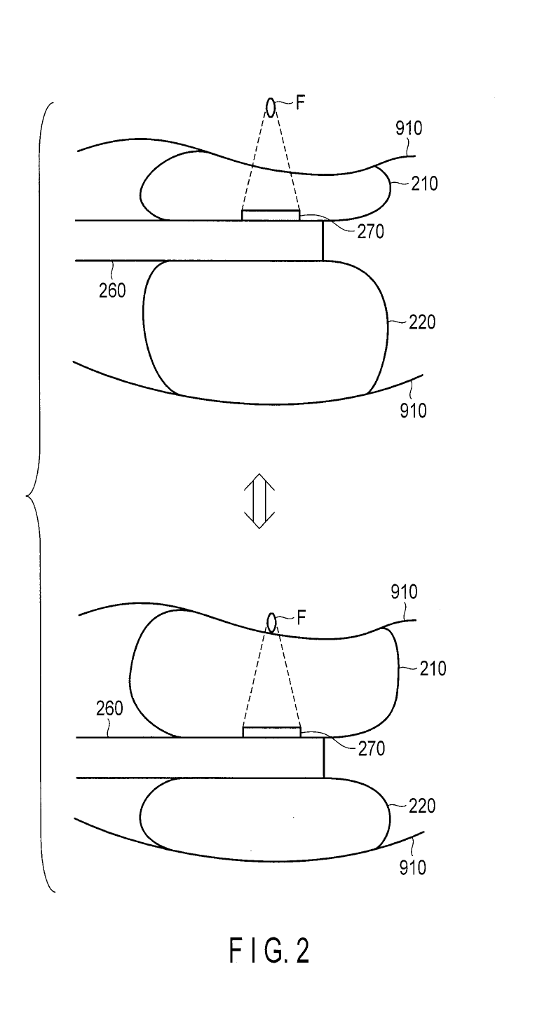

When the position to be irradiated with the ultrasonic wave is moved, for example, from the deep side toward the shallow side or from the shallow side toward the deep side, the user inputs an instruction to move the position, into the present ultrasonic irradiation device by use of the input section 120. The input section 120 into which the instruction to change the position to be irradiated with the ultrasonic wave has been input outputs this instruction to the control section 110. The control section 110 determines to what degrees the volume of the first balloon 210 and the volume of the second balloon 220 are to be changed, on the basis of the input instruction to change the position to be irradiated with the ultrasonic wave. That is, the control section 110 determines an increase or decrease amount of the volume of the first balloon 210, and an increase or decrease amount of the volume of the second balloon 220. For example, when the instruction to move the inserting portion 260 from the first balloon 210 side to the second balloon 220 side is input, as schematically shown in FIG. 2, the volume of the first balloon 210 is increased, and the volume of the second balloon 220 is decreased. The control section 110 outputs the determined volume change amounts of the first balloon 210 and the second balloon 220 to the pump control section 160.

The pump control section 160 controls the operations of the first pump 170, the second pump 175, the first three-way valve 180 and the second three-way valve 185 on the basis of the input volume change amounts of the first balloon 210 and the second balloon 220. For example, to move the inserting portion 260 from the first balloon 210 side to the second balloon 220 side, the first pump 170 is not operated, and the only second pump 175 may be operated to move the liquid of the second balloon 220 into the first balloon 210. When the size of the first balloon 210 is noticeably different from that of the second balloon 220, or when the movement amount of the inserting portion 260 is large and a total volume of the liquids included in the first balloon 210 and the second balloon 220 changes, the first pump 170 is operated as required. The first pump 170 may move a part of the liquid included in the first balloon 210 or the second balloon 220 into the liquid tank 190, or may refill the liquid from the liquid tank 190 into the first balloon 210 and the second balloon 220. In these operations, the pump control section 160 controls the operation of the second pump 175 so that the force of the liquid in the first balloon 210 to push the inserting portion 260 becomes equal to the force of the liquid in the second balloon 220 to push the inserting portion 260, and the liquid is fed.

Under the control of the pump control section 160, the first pump 170 and the second pump 175 move the liquid, to change the volumes of the first balloon 210 and the second balloon 220.

In this case, by an unshown ultrasonic probe for observation or an unshown position sensor, a distance between the inserting portion 260 and the wall surface 910 is measured on each of the first balloon 210 side and the second balloon 220 side, and the positional information is fed back to the control section 110 or the pump control section 160. This feedback control may be performed to dispose the inserting portion 260 at a predetermined position.

Furthermore, for example, when a treatment is ended and the probe part 200 is removed from, for example, the stomach, the user inputs an instruction to end the treatment into the input section 120. The input section 120 outputs, to the control section 110, the input instruction to end the treatment. On the basis of the instruction to end the treatment, the control part 100 outputs, to the pump control section 160, an instruction to contract the first balloon 210 and the second balloon 220. The pump control section 160 into which the instruction to contract the first balloon 210 and the second balloon 220 has been input operates the first pump to move the liquids from the first balloon 210 and the second balloon 220 into the liquid tank 190. As a result, the first balloon 210 and the second balloon 220 are contracted, and the fixed inserting portion 260 is released. Afterward, the user can remove the probe part 200 from the subject's living body.

As described above, according to the present embodiment, the inserting portion 260 of the ultrasonic irradiation device can be fixed to the inside of a space surrounded with the wall surface 910. As a result, the ultrasonic irradiation device can securely irradiate the target position with the focused ultrasonic wave. Moreover, the ultrasonic irradiation device can move the sound source 270 along an ultrasonic irradiating direction by changing the volume ratio between the first balloon 210 and the second balloon 220. That is, to cauterize an area having a size larger than that of the focal point of the focused ultrasonic wave in an ultrasonic wave travel direction in the ultrasonic irradiation device, the volume ratio between the first balloon 210 and the second balloon 220 is changed, so that while exactly changing a position of the area to be irradiated, the position can be irradiated with the ultrasonic wave.

It is to be noted that the first pressure sensor 240 does not have to be present in the first balloon 210, and may be disposed, for example, in the vicinity of the first three-way valve 180 of the first tube 230. Similarly, the second pressure sensor 245 may be disposed, for example, in the vicinity of the second three-way valve 185 of the second tube 235.

Moreover, in the description of the present embodiment, the embodiment has a configuration where the liquid to be filled into the first balloon 210 is the same as the liquid to be filled into the second balloon 220 and the liquids can mutually be moved, but separate liquids may be filled into the balloons, respectively. In this case, although not shown in the drawing, for example, a liquid contained in a liquid tank A may be injected into and discharged from the first balloon 210 by a pump A, and a liquid contained in a liquid tank B may be injected into and discharged from the second balloon 220 by a pump B. Furthermore, what is filled into the first balloon 210 and the second balloon 220 is not limited to the liquid, and may be a gas or a gel-like substance. However, the first balloon 210 functions as an ultrasonic propagating medium, and hence it is preferable that the substance to be filled into the first balloon 210 is, for example, physiological saline or deaerated water which has an acoustic impedance close to an acoustic impedance of a living organism as an ultrasonic irradiation target and has a small ultrasonic attenuation ratio. Moreover, when the present ultrasonic irradiation device is used in a living body, a substance harmless to the living body is used as the substance to be filled.

Furthermore, for emitting the focused ultrasonic wave, the ultrasonic emitting surface of the sound source 270 does not have to have the concave surface shape, and the ultrasonic wave may be converged by a phased array. That is, in place of the configuration where the sound source 270 includes one piezoelectric element, the sound source may have a configuration where piezoelectric elements are combined, for example, in a concentric manner. In this configuration, ultrasonic waves emitted from the respective piezoelectric elements may be converged by suitably regulating phases of the emitted ultrasonic waves. When the phased array is used, the ultrasonic irradiation device can change a focal point of the focused ultrasonic wave without changing the position of the sound source 270. Therefore, in the ultrasonic irradiation device, after changing the sizes of the first balloon 210 and the second balloon 220 to roughly change the position of the sound source 270, it is further possible to exactly regulate the focal point of the focused ultrasonic wave by the phased array. Furthermore, in the ultrasonic irradiation device, after roughly changing the focal point of the focused ultrasonic wave by the phased array, the position of the sound source 270 can exactly be regulated by further changing the sizes of the first balloon 210 and the second balloon 220. It is to be noted that the sound source 270 is not limited to the piezoelectric element, and may be any element as long as the element can emit the ultrasonic wave.

### Second Embodiment

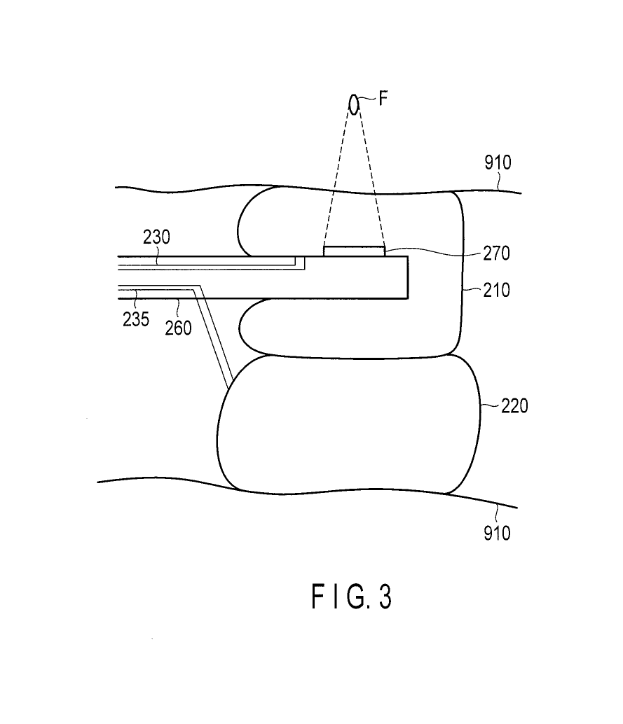

A second embodiment of the present invention will be described. Here, in the description of the second embodiment, a different part from the first embodiment will be described, and the same part is denoted with the same reference numerals, and the description thereof is omitted. In an ultrasonic irradiation device according to the present embodiment, positional relationships among an inserting portion 260, a first balloon 210 and a second balloon 220 of a probe part 200 are different from those of the ultrasonic irradiation device according to the first embodiment. A configuration of the probe part 200 according to the present embodiment is schematically shown in FIG. 3. In FIG. 3, for simplicity, a first pressure sensor 240, a second pressure sensor 245, a wiring line which connects these sensors to a pump control section 160, a wiring line which connects a sound source 270 to a sound source drive section 150 and the like are omitted and are not shown in the drawing, but are arranged similarly to the first embodiment.

As shown in FIG. 3, in the present embodiment, the first balloon 210 is disposed to surround the inserting portion 260. Furthermore, the second balloon 220 is disposed on a portion of the first balloon 210 which is positioned opposite to the sound source 270 via the inserting portion 260, on a side opposite to the inserting portion 260. The remaining configuration is similar to the configuration of the first embodiment.

Also in the present embodiment, a liquid is injected into and discharged from the first balloon 210 through a first tube 230, and the liquid is injected into and discharged from the second balloon 220 through a second tube 235. In this way, the first balloon 210 and the second balloon 220 can be expanded and contracted. Also in the present embodiment, a first pump 170, a second pump 175, a first three-way valve 180, a second three-way valve 185, a liquid tank 190 and the like in a control part 100 can be configured similarly to the first embodiment. In the configuration similar to the first embodiment, the liquid to be filled into the first balloon 210 is the same as the liquid to be filled into the second balloon 220.

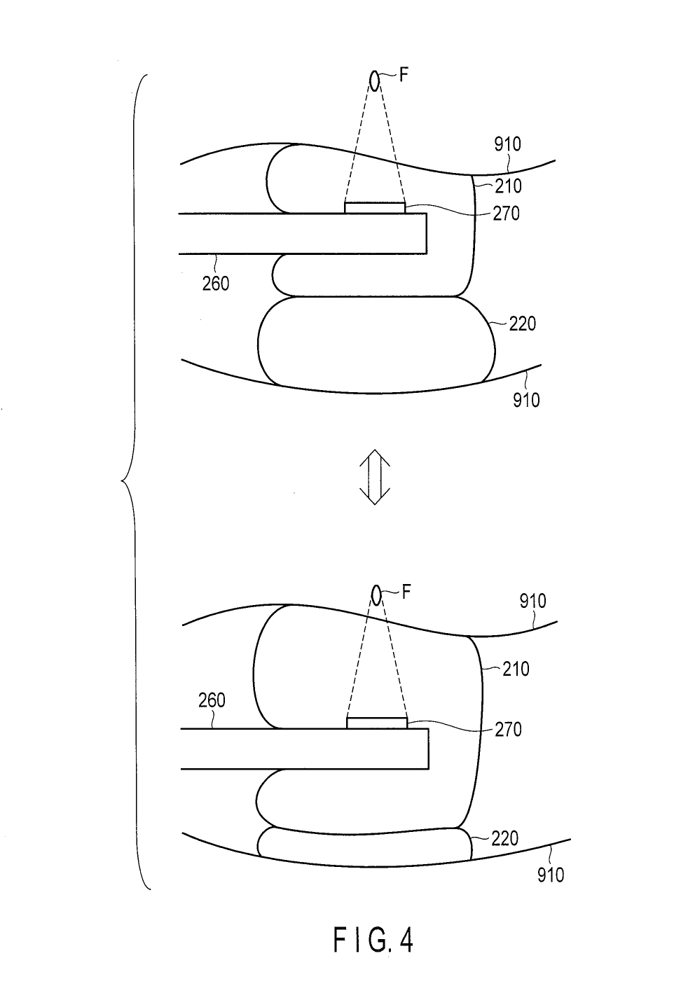

Also according to the present embodiment, volumes of the first balloon 210 and the second balloon 220 are regulated in the ultrasonic irradiation device similarly to the first embodiment, so that a tip of the inserting portion 260 can be fixed to a position intended by a user. Moreover, in the ultrasonic irradiation device, a position of the sound source 270 can be moved along an ultrasonic irradiating direction by changing a volume ratio between the first balloon 210 and the second balloon 220, as schematically shown in FIG. 4.

### Third Embodiment

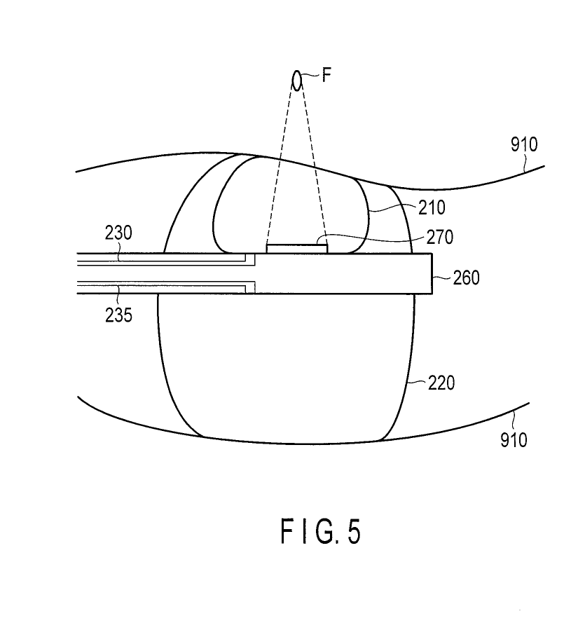

A third embodiment of the present invention will be described. Here, in the description of the third embodiment, a different part from the first embodiment will be described, and the same part is denoted with the same reference numerals, and the description thereof is omitted. In an ultrasonic irradiation device according to the present embodiment, positional relationships among an inserting portion 260, a first balloon 210 and a second balloon 220 of a probe part 200 are different from those of the ultrasonic irradiation device according to the first embodiment. A configuration of the probe part 200 according to the present embodiment is schematically shown in FIG. 5. In FIG. 5, similarly to FIG. 3, for simplicity, a first pressure sensor 240, a second pressure sensor 245, a wiring line which connects these sensors to a pump control section 160, a wiring line which connects a sound source 270 to a sound source drive section 150 and the like are omitted and are not shown in the drawing, but are arranged similarly to the first embodiment.

As shown in FIG. 5, in the present embodiment, the first balloon 210 is disposed on a sound source 270 side at a tip of the inserting portion 260. Furthermore, the second balloon 220 is disposed to cover the tip of the inserting portion 260 and the first balloon 210. Here, an outer surface of the first balloon 210 and an inner surface of the second balloon 220 are bonded to a portion where the first balloon 210 comes in contact with a wall surface 910. Moreover, the inserting portion 260 is formed to pass through the second balloon 220. The remaining configuration is similar to the first embodiment.

Also in the present embodiment, a liquid is injected into and discharged from the first balloon 210 through a first tube 230, and the liquid is injected into and discharged from the second balloon 220 through a second tube 235. In this way, the first balloon 210 and the second balloon 220 can be expanded and contracted in the ultrasonic irradiation device. Also in the present embodiment, a first pump 170, a second pump 175, a first three-way valve 180, a second three-way valve 185, a liquid tank 190 and the like in a control part 100 can be constituted similarly to the first embodiment. In the configuration similar to the first embodiment, the liquid to be filled into the first balloon 210 is the same as the liquid to be filled into the second balloon 220.

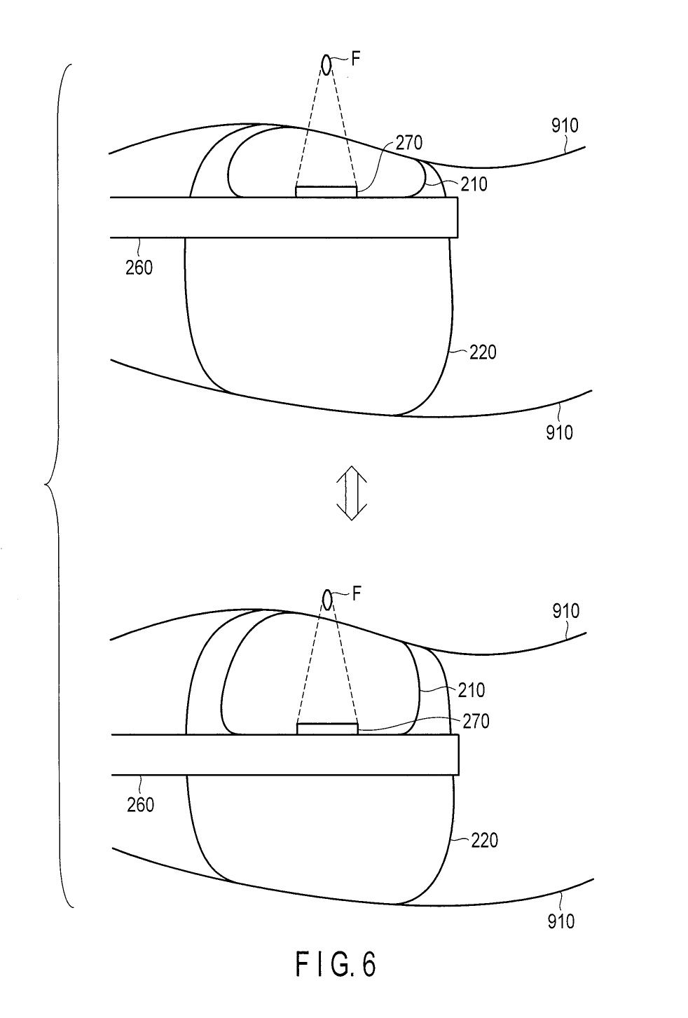

Also according to the present embodiment, volumes of the first balloon 210 and the second balloon 220 are regulated in the ultrasonic irradiation device similarly to the first embodiment, so that the tip of the inserting portion 260 can be fixed to a position intended by a user. That is, in the ultrasonic irradiation device, the second balloon 220 is expanded, to fix the whole second balloon including the inserting portion 260 and the first balloon 210 to the wall surface 910. In the ultrasonic irradiation device, a size of the first balloon 210 is regulated to adjust a distance between the sound source 270 and the wall surface 910. Moreover, in the ultrasonic irradiation device, a position of the sound source 270 can be moved along an ultrasonic irradiating direction by changing a volume ratio between the first balloon 210 and the second balloon 220, as schematically shown in FIG. 6.

### Fourth Embodiment

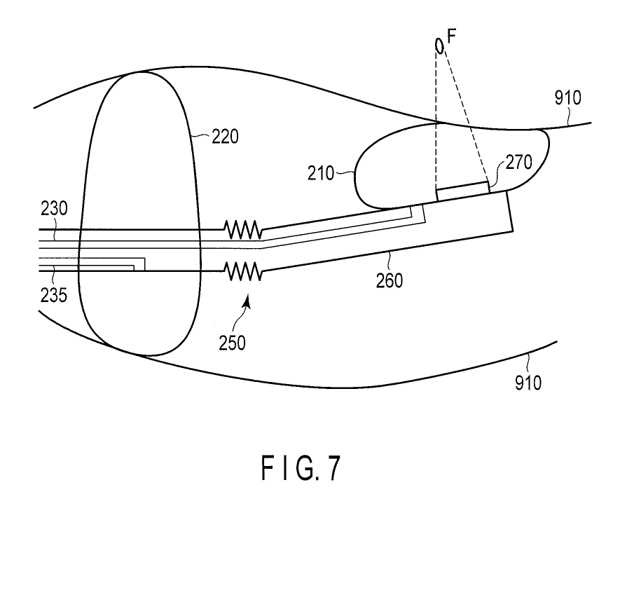

A fourth embodiment of the present invention will be described. Here, in the description of the fourth embodiment, a different part from the first embodiment will be described, and the same part is denoted with the same reference numerals, and the description thereof is omitted. In an ultrasonic irradiation device according to the present embodiment, a configuration of a probe part 200 is different from that of the ultrasonic irradiation device according to the first embodiment. The configuration of the probe part 200 according to the present embodiment is schematically shown in FIG. 7. In FIG. 7, similarly to FIG. 3, for simplicity, a first pressure sensor 240, a second pressure sensor 245, a wiring line which connects these sensors to a pump control section 160, a wiring line which connects a sound source 270 to a sound source drive section 150 and the like are omitted and are not shown in the drawing, but are arranged similarly to the first embodiment.

As shown in FIG. 7, in the present embodiment, the probe part 200 includes a joint portion 250 in the vicinity of a tip of an inserting portion 260 on a proximal side from the sound source 270. On the proximal side further from the joint portion 250, a second balloon 220 is disposed, and in this portion, the inserting portion 260 passes through the second balloon 220. The second balloon 220 can come in contact with a wall surface 910, and hence the proximal side of the inserting portion 260 from the joint portion 250 is fixed to the wall surface. The joint portion 250 is disposed as a support point, and a force is applied to the distal side of the inserting portion 260 from the joint portion 250, on the sound source 270 side by a spring mechanism of the joint portion 250. On the sound source 270 side at the tip of the inserting portion 260, a first balloon 210 is disposed. The first balloon 210 is pushed onto the wall surface 910 owing to an urging force of the above-mentioned spring mechanism of the joint portion 250. The remaining configuration is similar to the first embodiment.

Also in the present embodiment, a liquid is injected into and discharged from the first balloon 210 through a first tube 230, and a liquid is injected into and discharged from the second balloon 220 through a second tube 235. In this way, the ultrasonic irradiation device can expand and contract the first balloon 210 and the second balloon 220. Upon ultrasonic irradiation, the ultrasonic irradiation device regulates a volume of the second balloon 220, so that the proximal side of the inserting portion 260 from the joint portion 250 is fixed to the wall surface 910. The spring mechanism of the joint portion 250 pushes the inserting portion 260 toward the sound source 270. Therefore, in the ultrasonic irradiation device, a space between the sound source 270 and the wall surface 910 can be changed by adjusting a size of the first balloon 210.

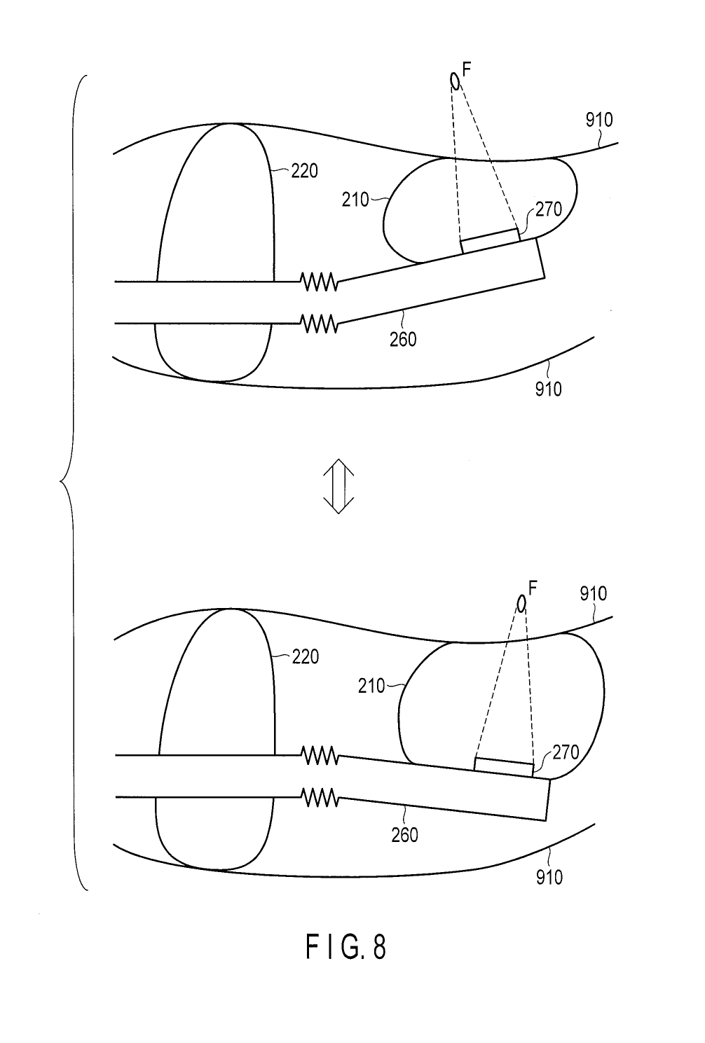

Also according to the present embodiment, similarly to the first embodiment, in the ultrasonic irradiation device, volumes of the first balloon 210 and the second balloon 220 are regulated, so that the tip of the inserting portion 260 can be fixed to a position intended by a user. That is, in the ultrasonic irradiation device, a proximal side of the inserting portion from the joint portion 250 can be fixed to the wall surface 910 by expanding the second balloon 220, and the space between the sound source 270 and the wall surface 910 can be regulated by the first balloon 210. Moreover, in the ultrasonic irradiation device, a position of the sound source 270 can be moved by changing the volume of the first balloon 210, as schematically shown in FIG. 8.

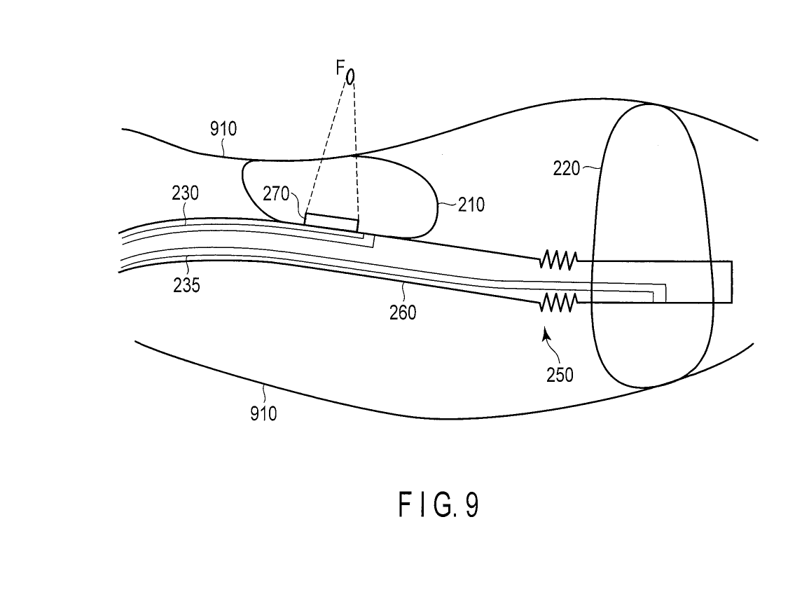

It is to be noted that as shown in FIG. 9, the second balloon 220 may be disposed on the distal side of the inserting portion 260, and the sound source 270 and the first balloon 210 may be arranged on the proximal side of the inserting portion via the joint portion 250. In this case, a portion of the inserting portion 260 between the joint portion 250 and the sound source 270 has a rigidity so that the first balloon 210 is pushed onto the wall surface 910 by the urging force of the spring mechanism of the joint portion 250, but the proximal side of the inserting portion 260 from a position of the inserting portion provided with the sound source 270 is constituted of a flexible material. Also in this case, an effect similar to that of the configuration described with reference to FIG. 7 can be obtained.

### Fifth Embodiment

A fifth embodiment of the present invention will be described. Here, in the description of the fifth embodiment, a different part from the first embodiment will be described, and the same part is denoted with the same reference numerals, and the description thereof is omitted. An ultrasonic irradiation device according to the present embodiment includes a third balloon 225 in addition to a first balloon 210 and a second balloon 220.

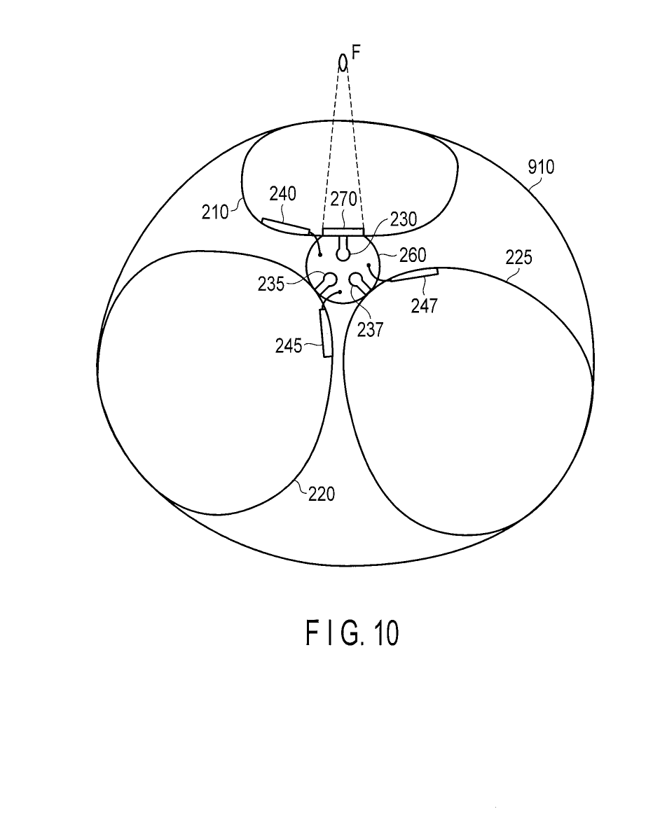

A configuration of a probe part 200 according to the present embodiment is schematically shown in FIG. 10. FIG. 10 is a view of an inserting portion 260 seen from a distal side thereof. As shown in FIG. 10, at a tip of the inserting portion 260, the first balloon 210, the second balloon 220 and the third balloon 225 are arranged to form an angle of 120 degrees with one another as seen from the distal side of the inserting portion 260. Here, the first balloon 210 is disposed on a sound source 270 side of the inserting portion 260. Similarly to the first embodiment, the first balloon 210 is connected to a first tube 230, and the second balloon 220 is connected to a second tube 235. Similarly to these balloons, the third balloon 225 is connected to a third tube 237. The first tube 230, the second tube 235 and the third tube 237 are connected to the first pump 170 and the second pump 175 via three-way valves.

Moreover, similarly to the first embodiment, a first pressure sensor 240 is disposed in the first balloon 210, and a second pressure sensor 245 is disposed in the second balloon 220. Similarly to these sensors, a third pressure sensor 247 is disposed in the third balloon 225. The first pressure sensor 240, the second pressure sensor 245 and the third pressure sensor 247 are connected to a pump control section 160, respectively. The remaining configuration is similar to the first embodiment.

Also in the present embodiment, a liquid is injected into and discharged from the first balloon 210 through the first tube 230, the liquid is injected into and discharged from the second balloon 220 through the second tube 235, and the liquid is injected into and discharged from the third balloon 225 through the third tube 237. Thus, in the ultrasonic irradiation device, it is possible to expand and contract the first balloon 210, the second balloon 220 and the third balloon 225, respectively.

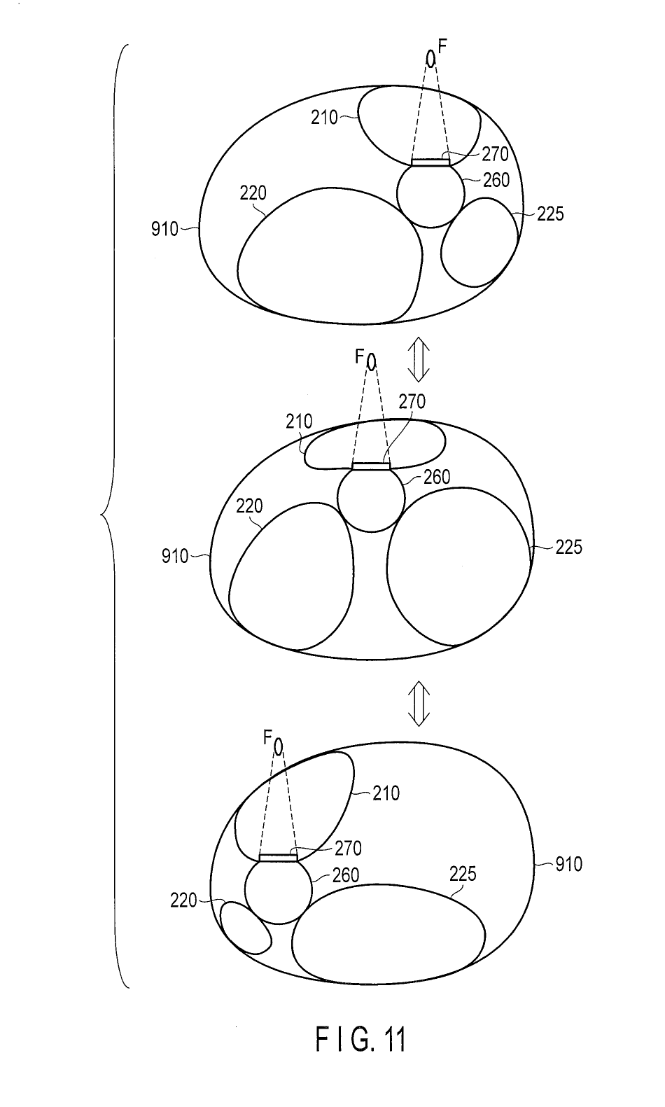

Also according to the present embodiment, similarly to the first embodiment, in the ultrasonic irradiation device, volumes of the first balloon 210, the second balloon 220 and the third balloon 225 are regulated, so that at a position intended by a user, the tip of the inserting portion 260 can be fixed to a wall surface 910. Moreover, in the ultrasonic irradiation device, a position of the sound source 270 can arbitrarily be moved by changing a volume ratio among the first balloon 210, the second balloon 220 and the third balloon 225, as schematically shown in FIG. 11. That is, when an area having a size larger than that of the focal point of a focused ultrasonic wave in an ultrasonic wave travel direction is cauterized by the ultrasonic irradiation device, a position of the area which is to be irradiated can exactly be changed by changing the volume ratio among the first balloon 210, the second balloon 220 and the third balloon 225. Furthermore, similarly to the present embodiment, the number of the balloons can be increased to four or more.

### Sixth Embodiment

A sixth embodiment of the present invention will be described. Here, in the description of the sixth embodiment, a different part from the first embodiment will be described, and the same part is denoted with the same reference numerals, and the description thereof is omitted. In the ultrasonic irradiation device according to the first embodiment, balloons are used to fix an inserting portion 260 to a wall surface. On the other hand, in the present embodiment, strut members using actuators are used in place of the balloons.

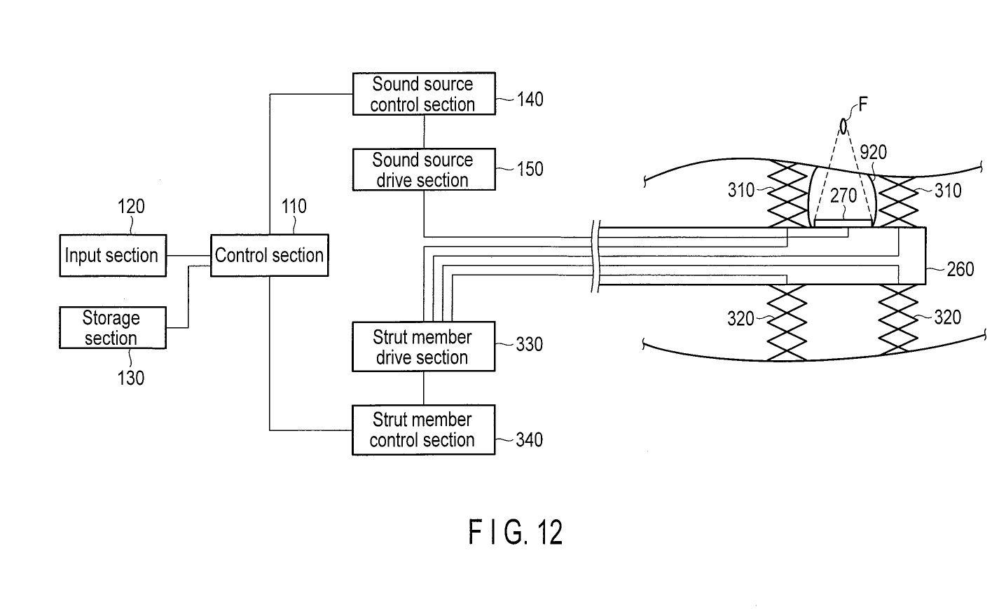

A configuration of the present embodiment is schematically shown in FIG. 12. An ultrasonic irradiation device according to the present embodiment includes a first strut member 310 in place of the first balloon 210 of the first embodiment, and includes a second strut member 320 in place of the second balloon 220. The first strut member 310 is disposed at a position where an ultrasonic wave emitted from a sound source 270 is not interrupted, on the sound source 270 side of an inserting portion 260. The second strut member 320 is disposed to face the first strut member 310 via the inserting portion 260. Here, the first strut member 310 and the second strut member 320 do not have to be arranged one by one, and as shown in, for example, FIG. 12, the members may be arranged, respectively.

The present ultrasonic irradiation device includes a strut member drive section 330 and a strut member control section 340, in place of the pump control section 160, the first pump 170, the second pump 175, the first three-way valve 180, the second three-way valve 185 and the liquid tank 190 of the first embodiment.

The first strut members 310 and the second strut members 320 are connected to the strut member drive section 330, and are driven by the strut member drive section 330 to extend and contract. The strut member drive section 330 is connected to the strut member control section 340. The first strut members 310 and the second strut members 320 which are driven by the strut member drive section 330 are controlled by the strut member control section 340. Moreover, the strut member control section 340 is connected to a control section 110.

In this way, for example, the first strut member 310 functions as a first support member. The second strut member 320 functions as a second support member. For example, the strut member control section 340 functions as a control section which changes a distance between a sound source and a target area.

An operation of the ultrasonic irradiation device according to the present embodiment will be described. A user inserts the inserting portion 260 of the present ultrasonic irradiation device, for example, from a subject's mouth through the esophagus into the stomach. At this time, the first strut members 310 and the second strut members 320 are contracted and received in the inserting portion 260. Therefore, a probe part 200 has a sufficiently slim configuration to pass through, for example, the esophagus. The user allows an ultrasonic emitting surface of the sound source 270 to face an area to be irradiated with a focused ultrasonic wave. In this state, an instruction to fix the probe part 200 is input into the present ultrasonic irradiation device by use of an input section 120.

The input section 120 receives the user's instruction to fix the probe part 200, and outputs the instruction to the control section 110. The control section 110 outputs, to the strut member control section 340, an instruction to start control for fixing the probe part 200. The instruction is input into the strut member control section 340 from the control section 110. The strut member control section 340 controls the strut member drive section 330 which drives the first strut members 310 and the second strut members 320. The first strut members 310 and the second strut members 320 are driven by the strut member drive section 330 to extend. When the first strut members 310 and the second strut members 320 push a wall surface 910 with predetermined pressures, the members stop the extending operation. Here, the first strut members 310 regulate a distance between the sound source 270 and the wall surface 910. The first strut members 310 and the second strut members 320 fix the inserting portion 260 to the wall surface 910. As a result, while maintaining a position of the inserting portion 260 inserted by the user, the inserting portion 260 is fixed to the wall surface 910 by the first strut members 310 and the second strut members 320.

On confirming that a position to be irradiated with the focused ultrasonic wave is superimposed on a focal point F, the user inputs, into the input section 120, an instruction for ultrasonic irradiation. Afterward, similarly to the first embodiment, the sound source 270 of the present ultrasonic irradiation device emits the focused ultrasonic wave. When the ultrasonic irradiation device according to the present embodiment is used, to enhance a propagating efficiency of the ultrasonic wave, an ultrasonic propagating medium 920 is preferably separately interposed between the sound source 270 and the wall surface 910.

Moreover, for example, when the ultrasonic irradiating position is moved, the user inputs an instruction to move the position by use of the input section 120. At this time, the input section 120 into which the user's instruction to change the position to be irradiated with the ultrasonic wave has been input outputs the instruction to the control section 110. On the basis of the input instruction to change the position to be irradiated with the ultrasonic wave, the control section 110 instructs the strut member control section 340 to move the inserting portion 260. The strut member control section 340 controls the strut member drive section 330 to extend and contract the first strut members 310 and the second strut members 320. For example, when the focal point F of the focused ultrasonic wave is moved from the inside toward the wall surface, the first strut members 310 are extended, and the second strut members 320 are contracted. As a result, the position of the inserting portion 260 to the wall surface 910 changes.

Moreover, for example, when a treatment is ended and the probe part 200 is removed from, for example, the stomach, the user inputs an instruction to end the treatment into the input section 120. The input section 120 outputs the input instruction to end the treatment to the control section 110. On the basis of the instruction to end the treatment, the control section 110 outputs, to the strut member control section 340, an instruction to contract the first strut members 310 and the second strut members 320. The strut member control section 340 controls the strut member drive section 330 to contract the first strut members 310 and the second strut members 320. As a result of the contraction of the first strut members 310 and the second strut members 320, the fixed inserting portion 260 is released. Afterward, the user can remove the probe part 200 from the subject's living body.

As described above, according to the present embodiment, the ultrasonic irradiation device can fix the inserting portion 260 to the space surrounded with the wall surface 910. In consequence, the ultrasonic irradiation device can securely irradiate the target position with the focused ultrasonic wave. Moreover, the ultrasonic irradiation device can move the position of the sound source 270 by changing lengths of the first strut members 310 and the second strut members 320.

A shape of each of the first strut members 310 and the second strut members 320 shown in FIG. 12 is an example for explaining the present embodiment, and the shape may be any shape as long as the inserting portion 260 can be fixed to the wall surface 910. For example, the first strut members 310 and the second strut members 320 may have a stent-like shape.

Moreover, in the present embodiment, the first strut members 310 are arranged in place of the first balloon 210 of the first embodiment, and the second strut members 320 are arranged in place of the second balloon 220 of the first embodiment, but the first strut members 310 may be arranged in place of the first balloon 210 of the fourth embodiment, and the second strut members 320 may be arranged in place of the second balloon 220 of the fourth embodiment. In this case, the ultrasonic irradiation device operates in the same manner as in the fourth embodiment, and a similar effect can be obtained.

Furthermore, the strut members may similarly be arranged in place of the balloons in the fifth embodiment.

It is to be noted that various inventions can be formed by suitably combining constituent elements disclosed in the above embodiments. For example, the first balloon 210 of the first embodiment may be used as the first support member, and the second strut members 320 of the seventh embodiment may be used as the second support member. Moreover, in any one of the embodiments, a sound source which can converge an ultrasonic wave by a phased array can be used as the sound source 270.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic irradiation device comprising:
a sound source configured to be placed in a space at least a part of which is covered with a wall surface and emit an ultrasonic wave toward a target area;
a holding member configured to hold the sound source;
a first support member configured to push a first area of the wall surface so that a distance between the sound source and the target area is kept at a predetermined value, the first supporting member being disposed on the holding member; and
a second support member configured to push a second area of the wall surface including an area other than the first area so that the sound source is fixed to the wall surface together with the first support member.

2. The ultrasonic irradiation device according to claim 1, wherein
the first support member is disposed to face the second support member via the holding member.

3. The ultrasonic irradiation device according to claim 2, wherein
the first support member is disposed on a side of the sound source facing the target area.

4. The ultrasonic irradiation device according to claim 1, wherein
at least one of the first support member and the second support member includes a balloon whose size changes in accordance with an amount of a fluid to be filled into the balloon.

5. The ultrasonic irradiation device according to claim 1, wherein
the first support member includes a first balloon whose size changes in accordance with an amount of a first fluid to be filled into the first balloon, and
the second support member includes a second balloon whose size changes in accordance with an amount of a second fluid to be filled into the second balloon.

6. The ultrasonic irradiation device according to claim 5, wherein
a composition of the first fluid is the same as that of the second fluid.

7. The ultrasonic irradiation device according to claim 1, wherein
the first support member includes a first balloon whose size changes in accordance with an amount of a first fluid to be filled into the first balloon,
the second support member includes a second balloon whose size changes in accordance with an amount of a second fluid to be filled into the second balloon,
the first balloon and the sound source are arranged in the second balloon, and
in a portion of the first support member which pushes the first area, an outer surface of the first balloon comes in contact with an inner surface of the second balloon.

8. The ultrasonic irradiation device according to claim 7, wherein
a composition of the first fluid is the same as that of the second fluid.

9. The ultrasonic irradiation device according to claim 1, wherein
the holding member has a rod shape and includes a bendable joint portion including a spring mechanism,
the sound source is disposed on a part of a peripheral surface of the holding member having the rod shape,
the first support member is disposed on a side of the sound source facing the target area,
the second support member is disposed on a side of the holding member opposite to a side provided with the sound source via the joint portion in a longitudinal direction of the holding member, and
the spring mechanism applies a force so that the side of the holding member provided with the sound source via the joint portion is displaced in a direction toward the target area.

10. The ultrasonic irradiation device according to claim 1, further comprising:
a control section which deforms the first support member and the second support member in conjunction with each other, to change the distance between the sound source and the target area.

11. The ultrasonic irradiation device according to claim 5, further comprising:
a fluid adjusting section which changes the amount of the first fluid to be filled into the first balloon and the amount of the second fluid to be filled into the second balloon; and
a control section which controls the fluid adjusting section to change the size of the first balloon and the size of the second balloon, thereby changing the distance between the sound source and the target area.

12. The ultrasonic irradiation device according to claim 9, wherein
the first support member includes a first balloon whose size changes in accordance with an amount of a first fluid to be filled into the first balloon, and
the second support member includes a second balloon whose size changes in accordance with an amount of a second fluid to be filled into the second balloon,
the ultrasonic irradiation device further comprising:
a fluid adjusting section which changes the amount of the first fluid to be filled into the first balloon, and
a control section which controls the fluid adjusting section to change the size of the first balloon, thereby changing the distance between the sound source and the target area.

13. The ultrasonic irradiation device according to claim 1, comprising a plurality of the second support members.

14. The ultrasonic irradiation device according to claim 13, wherein
the first support member includes a first balloon whose size changes in accordance with an amount of a first fluid to be filled into the first balloon, and
the second support members include second balloons whose sizes change in accordance with an amount of a second fluid to be filled into each of the second balloons.

15. The ultrasonic irradiation device according to claim 1, wherein
the sound source is configured to change a focal point of the emitted ultrasonic wave by using a phased array.

16. The ultrasonic irradiation device according to claim 5, wherein
the first balloon is disposed on a side of the sound source facing the target area, and
the first balloon is filled with an ultrasonic propagating medium to match an acoustic impedance of the sound source with that of the target area.
